(19) United States
(12) Patent Application Publication
(10) Pub. No.: US 2023/0145284 A1
(43) Pub. Date: May 11, 2023

(54) APPARATUS FOR TREATMENT OF THE RETINA BY RADIATION

(71) Applicant: CIROMED GMBH, Köln (DE)

(72) Inventor: GEORG GERTEN, Bonn (DE)

(73) Assignee: CIROMED GMBH, Köln (DE)

(21) Appl. No.: 17/918,603

(22) PCT Filed: Apr. 7, 2021
(86) PCT No.: PCT/EP2021/059089

§ 371 Date: Oct. 13, 2022

(30) Foreign Application Priority Data
Apr. 15, 2020 (DE) 10 2020 110 284.4

(51) Int. Cl. A61N 5/06

(52) U.S. Cl. CPC A61N 5/0622; A61N 2005/063; A61N 2005/0648; A61N 2005/0659; A61N 2005/0663; A61N 2005/0626

(57) ABSTRACT

A device for therapeutic treatment of the retina includes a holding and positioning device for holding an optical lens element in a defined position and orientation in front of the eye of a patient, the lens element having a irradiation-transmitting concave surface for resting on the closed eyelid, and the irradiation source cooperating with the optical lens element to deliver the therapeutically effective irradiation to the retina via the optical lens element.

### CROSS-REFERENCE TO FOREIGN PRIORITY APPLICATION

The present application claims the benefit under 35 U.S.C. §§ 119(b), 119(e), 120, and/or 365(c) of PCT/EP2021/059089 filed Apr. 7, 2021, which claims priority to German Application No. DE 10 2020 110 284.4 filed Apr. 15, 2020.

### FIELD OF THE INVENTION

The invention relates to a device for therapeutic treatment of the retina by means of photobiomodulation using a dosed irradiation treatment.

### BACKGROUND OF THE INVENTION

It is scientifically known and used in therapeutic approaches for the treatment of degenerative diseases and other pathological changes of tissue to treat human tissue by exposure to irradiation. In addition to irradiation with hard rays, such as radiotherapeutic irradiation of tumour cells, which is not considered in detail here and is not associated with the invention, it has recently become known that irradiation with irradiation of a wavelength in the visible light range and in wavelength ranges adjacent thereto can also achieve therapeutic effects. Irradiation is used for different tissues, for example for wound healing of skin tissue and subcutaneous tissue.

A special field of application is the treatment of the eye, more precisely the retina, by means of such irradiation in the visible light range and the adjacent electromagnetic irradiation, (wavelengths from 400 nm to 1500 nm are used here). A dosed irradiation treatment can be used for the therapy of degenerative diseases of the retina. The cells of the retina are naturally sensitive to light irradiation and particularly sensitive to an incorrect dosage of irradiation. According to the inventor's knowledge, in addition to the relationship between the duration of exposure to optical irradiation and the irradiation intensity, wavelength range, presentation mode (e.g., pulsed or continuous irradiation, angle of incidence, and irradiation area of the irradiation), the repetition rate and the time of exposure (circadian rhythm) are also factors that can positively or adversely influence the desired therapeutic effect, or even change it into a cell-damaging effect. The human retina in particular is heavily supplied with mitochondria, which can have a pronounced circadian metabolic activity and, therefore, according to the inventor's knowledge, have different sensitivities to certain frequencies of electromagnetic irradiation in such therapeutic treatments at different times of the day. The susceptibility of cellular structures to electromagnetic irradiation may also vary depending on the age of the individual, their individual constitution or the disease. Furthermore, the retina is neuronally complexly interconnected (e.g., in the form of so-called on-off fields), so that irradiations of certain areas can have an influence on non-irradiated or less irradiated areas.

From U.S. Pat. No. 10,219,944 B2 and US2016/0067087 A1 a device is known which enables a treatment of the retina of the eye with a predetermined treatment programme. For this purpose, a special treatment device is provided which fixes the head of the user in a defined position, then fixes the position of the eye and the orientation of the head in a defined manner by resting on a fixing frame section of the device, in order to then carry out irradiation of the retina of both eyes for therapy purposes. However, the disadvantage of this device is that a large number of treatment procedures are necessary for the therapeutic effect and, although the device can guarantee the necessary safety for carrying out the treatment due to its special construction, it is not suitable for being available to the patient himself for one treatment procedure—for example, by the patient purchasing or borrowing it himself—due to its special construction and the associated manufacturing costs. The patient must therefore go to an inpatient or outpatient facility on a recurring basis for a large number of treatment procedures, for which the acquisition of such a device can be economically represented due to a high utilisation rate that can be achieved there.

In principle, it is desirable both to reduce the effort involved for the patient and to avoid the high costs that this entails for the health care system. In principle, one approach to avoiding therapy costs through outpatient or inpatient treatment has always been to enable patients to carry out their own therapy. However, on the one hand, this cannot be done with cost-intensive devices, since the acquisition of such devices would run counter to the desired cost-saving effect. On the other hand, such treatments, which can cause tissue damage instead of the intended therapy due to incorrect operation, faulty settings, or the like, are regularly not suitable for home use.

The irradiation device known from U.S. Pat. No. 10,219,944 B2 and US2016/0067087 A1 for the setting up and defined positioning of the patient's head is a device suitable for use in inpatient or outpatient treatment centres, the acquisition of which is well suited for use by a large number of patients in spaced, longer intervals of the treatment procedures. However, for economic reasons as well as due to the adjustment and support work required for these devices, a doctor must accompany the patient during the treatment procedure and the device is therefore not suitable for a cost-saving and at the same time technically and medically safe treatment procedure over a longer period of time with several treatment procedures at intervals.

Another device for irradiation treatment of the retina is known from WO 2018/224671. This device is a system consisting of, on the one hand, a specially made device which is worn by the patient in the manner of a spectacle frame. Irradiation sources are attached to the spectacle frame and are controlled by a separate control unit belonging to the system for the therapy treatment. This device is also a special device consisting of two treatment devices to be purchased, on the one hand the special control device, on the other hand the special spectacle-like treatment device with irradiation source. Here, too, an economically efficient and at the same time medically safe implementation of a long-term therapy with several temporally spaced treatment procedures is therefore not feasible.

An irradiation device for therapy of the human eye is also known from US2016/0158486 A1. The devices taught in this document are characterised by a great variety of designs, so that on the one hand the device can be worn close to the eye or in the eye. Furthermore, it is explained that contact with the eyelid is also conceivable as an embodiment. However, a disadvantage of this previously known teaching is that there is no information about the arrangement of the components which are necessary for the construction and for an effective effect of the device. In addition, the device is based on a distant transmission of the irradiation and thus achieves an unfavourable transmission rate and has to accept unfavourable thermal effects.

Another irradiation device is known from US 2013/0304162 A1, which is to be worn in the manner of ski goggles. Here, a distant irradiation transmission through the air into the open eye is relied upon, which on the one hand results in a dependence on eye movements that are unavoidable in practice and, on the other hand, an only imprecise dosage of the irradiation intensity and the total irradiation dose can be achieved by closing the eyelids.

From US 2017/0333729 A1 and US 2019/0232078 A1 beta irradiation devices for both eyes with a structure similar to a sleep mask are known. These devices do not provide a reliable dosage of the irradiation intensity nor a reliable dosage of the total irradiation dose and are therefore suitable as a means for an accompanying measure of a therapy, but cannot be used as therapy-effective irradiation devices, as they do not guarantee the necessary precision neither with regard to the irradiation direction nor the irradiation dosage.

An irradiation device for the eye is known from US 2020/008135 A1, which is based on the principle of transmission through the air from a plurality of light sources with a diffuse alignment of the irradiation. With this irradiation device, a therapeutically targeted treatment of diseased tissue areas is not possible and the device is altogether too bulky and unwieldy to be carried comfortably by the user and for a longer treatment period.

A technical problem in the treatment of irradiation-sensitive tissues with therapeutic irradiation is that too high an exposure to irradiation can cause damage to the tissue instead of the intended therapeutic effect. Such a too high irradiation exposure can be caused on the one hand by a too high irradiation intensity of the irradiation source or by a too short distance between irradiation source and retina by a too long exposure or by a too short repetition rate of the exposure or by a combination of these causes. As a rule, such therapeutic irradiation treatment can therefore be carried out with special devices that reliably prevent these causes of damage, i.e., maladjustment, faulty operation, or control, and is often only approved for treatment by corresponding approval authorities if corresponding safety devices are available on the device. On the other hand, if certain operating errors cannot be ruled out by the device itself, medical supervision is regularly necessary during therapy. Both effects are disadvantageous for an economic implementation of treatments that extend over a long period of time with several treatment procedures that are spaced apart in time, and pose a problem if a device is to be technically developed that is more economical and at the same time can be used in a safe manner.

### SUMMARY OF THE INVENTION

Against this background, it is a task of the invention to provide a device system for the radiotherapeutic treatment of retinal tissue which, more economically than the previously known systems, can carry out a long-term treatment with several treatment procedures spaced apart in time and at the same time can reliably avoid technical safety for the avoidance of incorrect treatments which lead to tissue damage.

This task is solved by the invention by providing a device which has the features disclosed herein.

According to the invention, a device is provided which is used for therapeutic treatment of the retina with irradiation. The device comprises on the one hand a irradiation source, and on the other hand a control unit which controls this irradiation source. This control serves primarily to control a irradiation duration and a irradiation intensity. However, depending on the treatment method, other parameters, such as irradiation frequencies and irradiation spectra, can also be controlled. Irradiation source and control unit can be combined in one housing or arranged separately. The signal transmission can be wireless or wired.

The invention is fundamentally based on the realisation that an irradiation treatment of the retina, i.e., a photobiomodulation (PBM), unfolds an optimal effect, and may even only be effective if it is adapted to the metabolic situation of the target tissue, i.e., the corresponding cells of the retina to be addressed. In the therapy of age-related macular degeneration (AMD), the circadian rhythm of the mitochondria must be taken into account above all. PBM therapy for AMD is particularly effective, or even only effective, if the irradiation is administered in the morning and preferably daily. In principle, this is realistic and feasible only if it is done at home. The therapy device according to the invention serves to provide patients with a daily morning irradiation dose. This embodiment is particularly suitable for use on a lying patient, so that the patient can perform the therapy in the morning before getting up. PBM therapy of bedridden patients or generally ill patients is also possible.

The device according to the invention comprises a holding and positioning device. This holding and positioning device serves to position an optical lens element, which is part of the invention and is connected to this holding and positioning device, in front of the eye of a patient. The objective element is used to introduce therapeutic irradiation into the eye of the patient. For this purpose, the objective element must be held in a specific position and in a specific orientation in front of the patient's eye and this position and orientation must be reliably maintained during the treatment. Devices are known from the prior art for this purpose, which position the patient's head as a whole in order to achieve such alignment and positioning of the irradiation. Auxiliary devices similar to goggles are also known, which are intended to ensure a certain axial alignment and position of the irradiation. However, these devices have shown to be disadvantageous in that a safe irradiation of the retina, for example, also for home use by the patient without supervision by medically trained personnel, is not possible with the necessary safety. In particular, the individual positioning and alignment of the patient is problematic due to different distances between the eyes, a possible defective vision and, due to the problem of maintaining an approximately axially accurate positioning and alignment in relation to the visual axis for both eyes by means of a frame attached to the patient's head for the entire duration of treatment.

This problem is overcome with the device according to the invention. On the one hand, the holding and positioning device serves to hold the lens element in a defined position and orientation in front of the eye. On the other hand, the lens element has a radiolucent concave surface that is adapted to the convex surface of the eye. On the one hand, this adaptation may serve to allow the concave surface to rest directly on the patient's eye, preferably the concave surface is geometrically adapted to rest on the closed surface of the eyelid. During treatment, this concave surface ensures reliable positioning and alignment of the lens element as a first effect. At the same time, as a second effect, an impact of the irradiation is also achieved by this direct support through the irradiation-effective coupling into the eye or eyelid. The contact surface of the concave surface of the lens element can preferably adapt to the surface shape of the eyelid. The shape of the eyelid surface varies slightly from patient to patient and also changes intra-individually depending on the viewing direction of the eye below the closed eyelid. This adaptability can be realised, for example, by an elasticity of the lens element itself, such as in the manner of a soft contact lens. The adaptability can also be realised with a rigid lens element and an elastic intermediate layer in the manner of a gel or the like.

It is also advantageous that the concave contact surface of the lens element allows temperature conduction between the eyelid and the lens element, which compensates for unpleasant heating due to irradiation and makes treatment much more pleasant for the patient.

The device according to the invention thus achieves the decisive advantage over known devices which aim to achieve irradiation directly through the pupil opening and eye lens when the eyelid is open, in that more precise positioning and alignment as well as more favourable irradiation coupling into the eye is achieved due to the direct mechanical support of the lens element on the eyelid.

The at least one irradiation source can emit therapeutically effective irradiation with a irradiation intensity of more than 0.01 mW/cm² (=0.1 W/m²), more than 0.025 mW/cm², more than 0.05 mW/cm², more than 0.2 mW/cm² or more than 0.5 mW/cm², which provides a sufficient irradiation dose for treatment with the eyelid open. It is particularly preferred if the irradiation source emits irradiation with an irradiation intensity of more than 0.1 mW/cm², which is particularly suitable for treatment through the closed eyelid. In this context, it should be understood that the irradiation intensity in each case is understood to be the irradiation intensity present from the exit surface of the device in the exit surface, which can still attenuate or intensify through divergence or convergence up to the retina—and, in particular, can still be reduced through absorption effects in the eyelid or in other tissue parts lying between the exit surface and the retina.

In principle, a therapeutically effective treatment can be achieved with these lower limits of irradiation intensity. The irradiation intensity preferably remains below 0.1 mW/cm², below 0.5 mW/cm² or below 10 mW/cm², in particular below 1 mW/cm² or 5 mW/cm², with an irradiation duration of up to 100 s, whereby the permissible irradiation effect both for the eye itself and for the skin of the eyelid is not exceeded. It should be understood that the relationship between permissible irradiation duration and permissible intensity level follows the usual scientific criteria.

The at least one irradiation source can emit therapeutically effective irradiation in the wavelength range from 450 to 1500 nm, preferably in four different wavelength ranges within this spectrum. These four different wavelength ranges can be between 450 and 500 nm, between 570 and 600 nm, between 610 and 730 nm and between 790-890 nm and can be applied either simultaneously or with a time delay. In particular, only two ranges can be used as core wavelengths, preferably the ranges around 670±15 nm and around 810±15 nm as well as around 850±15 nm.

The at least one irradiation source can emit irradiation with a power between 1 μW and 10 W, preferably between 100 μW and 1 W, and ideally between 1 mW and 100 mW. Here, the power is to be understood as the output power of the irradiation source itself, for example, of the diodes used as the irradiation source.

The lens element may consist of several optically active elements or be formed by a single optically active element. The concave surface of the lens element is adapted to the geometry of the eye or closed eyelid and therefore typically has a radius that is between 5 and 500 mm, ideally between 10 and 100 mm. The concave surface may be composed of several segments with different radii or may include planar sections. As previously explained, adaptability of the surface to different radii is preferred. In particular, the concave surface of the objective element may also have a geometry and radius such that a hygienic film or hygienic release liner, which may be used as a reusable or disposable release liner, may be attached thereto to enable good product hygiene and sterilisability of the body contact surfaces of the device.

In the preferred embodiment that the concave surface is designed to rest on the closed eyelid, the patient also has no relevant reason to make eye movements during the treatment due to the closed eyelid, so that a therapeutically effective and reliable irradiation is achieved. These advantages outweigh the disadvantage of a certain attenuation and filtering of the irradiation through the closed eyelid and achieve an overall safer and more reliable method of treatment, which is particularly suitable for home use by the patient.

In accordance with an alternative or preferred aspect of the invention, the optical lens element comprises a diffuser configured to distribute irradiation from the irradiation source onto the irradiation-transmissive concave surface. According to this embodiment, the lens element comprises a diffuser. This diffuser may be formed directly by an element resting on the eye or eyelid surface, which constitutes the lens element itself or which is part of the lens element. However, the diffuser can also be arranged elsewhere in the lens element. In this context, a diffuser is understood to be an optically effective element which achieves a distribution effect by, for example, optical scattering or refraction. This can be, for example, a scattering element, hologram, lens, or light guide as an optical element. A typical example of such a diffuser effect is an optical effect similar to a frosted glass pane, such as is achieved by transparent materials with a crystalline or semi-crystalline structure. The diffuser effect achieves a distribution of the irradiation that is favourable for therapeutic treatment. On the one hand, this distribution can be designed in such a way that a larger irradiation surface is generated from a punctual and/or directed irradiation and/or a diffusion in the sense of an undirected irradiation, but possibly with a preferential direction, is generated from it.

It is even more preferred if the diffuser is designed as an integrating sphere or as a layer of a semitransparent material, in particular, polytetrafluoroethylene. According to the knowledge of the inventor, these specific embodiments of the diffuser have proven to be particularly suitable for integration into the device according to the invention and achieve a diffusion effect which is advantageous for therapeutic purposes.

It is further preferred if the lens comprises a thermal heating or cooling device. By means of such a heating or cooling device, a temperature of the contact surface that is comfortable for the patient can be achieved on the one hand, and temperature outflow from the eye or eyelid and corresponding temperature inflow can be avoided on the other hand. In particular, the heating or cooling device can be connected to a control unit which sets a constant temperature in the eyelid or in the lens element in order to compensate for temperature influences occurring during the treatment and to avoid heating of the lens element through irradiation.

A further embodiment of the invention comprises a set of contact surface elements, each of which is attachable to the concave surface of the lens element, the set comprising: a first contact surface element having a first refractive index, and a second contact surface element having a second refractive index different from the first refractive index. With this further development, the possibility is created that the refractive index of the lens can be changed and thus adapted to the tissue material being irradiated. This has proven to be particularly advantageous in order to achieve effective coupling into the closed eyelid and thereby avoid excessive heating of the lens element. The contact surface elements can be designed as foils that can adhere adhesively to an exit surface of the lens element.

It is even more preferred if the holding and positioning device is designed as a partial face mask which at least partially covers the eye area of the face. Such a design of the holding and positioning device enables on the one hand a comfortable wearing of the device according to the invention by the patient, but on the other hand also establishes an accuracy sufficient for the purposes of positioning and alignment, which, in particular, allows a precise alignment and positioning based thereon by means of the concave contact surface of the lens element. In particular, it is preferred here if a first basic positioning and alignment of the lens element is achieved by the holding and positioning device, but the lens element itself is still mounted in the holding and positioning device so as to be movable within limits, in order to enable a second, precise alignment and positioning on the basis of the form-fit effect of the concave surface for the lens element itself.

In particular, the partial face mask can also be designed in such a way that it rests on the skin surface adjacent to the eyes and here achieves a basic form-fit positioning by means of the bone contours under the skin surface, as is known, for example, for spectacle frames.

In particular, it is further preferred if the partial face mask is held in a predetermined position on the patient's face by means of a strap guided around the head or by means of holding elements guided around the ears, such as straps or spectacle straps, or by means of suction cups. By means of these fastening measures and positioning means, a good fixation and retention on the patient's face sufficient for a basic alignment and positioning is achieved.

According to a further preferred embodiment, it is provided that the irradiation source is attached to the holding and positioning device, in particular, lies in a treatment position in a irradiation axis with the pupil and the diffuser. According to this embodiment, the irradiation device is directly arranged and fixed to the holding and positioning device. The patient can therefore comfortably wear this holding and positioning device and does not need a connection to an external device located outside the face. The irradiation source is here preferably located in an irradiation axis with the pupil and the diffuser in order to achieve direct irradiation propagation without deflection in the direction of the retina along an axis.

It is further preferred if the device is further formed by arranging a second irradiation source at a distance from the holding and positioning device on a irradiation base device and guiding the irradiation by means of a flexible irradiation guide from the irradiation base device to the holding and positioning device. According to this embodiment, in addition to the irradiation source arranged directly on the holding and positioning device, an additional second irradiation source is also provided, which is spaced apart from the holding and positioning device. The irradiation of this second irradiation source is conducted to the objective element by means of an irradiation conductor, for example a glass fibre line. The therapeutic irradiation is composed of the first and second irradiation sources, whereby these can act simultaneously in an additive manner on the retina or can also act alternately.

According to an alternative embodiment, the irradiation source is arranged at a distance from the holding and positioning device on an irradiation base unit and the irradiation is guided from the irradiation base unit to the holding and positioning device by means of a flexible irradiation guide. According to this embodiment, the irradiation source is not arranged and fixed to the holding and positioning device, so that the holding and positioning device can be designed as a light and compact unit. Instead, the irradiation source is arranged on an irradiation base unit that is spaced apart from the holding and positioning device, for example, in the form of a table-top unit, and that the patient does not have to attach to or carry on the body. From this irradiation base unit, the therapeutic irradiation is then guided to the objective element by means of an irradiation guide. Such an irradiation guide can be designed as a single or multi-core light guide arrangement, and, in particular, separate irradiation guides can also be provided in order to irradiate the left and right eye and to apply irradiation to two separate lens elements for the left and right eye accordingly. On the one hand, this enables an efficient dosage of the irradiation power that is sent via the irradiation conductors, and, on the other hand, an individual exposure of the two eyes of the user to a possibly different therapeutic irradiation.

According to a further preferred embodiment, the concave surface is designed to lie flat on the eye or the closed eyelid over a contact area of at least 1 cm2. Through this design, a flat support is achieved over a minimum area of 1 cm2 through the concave support surface. This ensures a favourable coupling of therapeutically effective irradiation over a sufficiently large area and avoids an unfavourable irradiation effect due to possibly high irradiation concentrations. At the same time, a secure, positive positioning of the objective element is achieved by the contact surface of this minimum size, which is also advantageous for the exact alignment and positioning of the irradiation axis.

The device can be further advanced by an eyelid sensor signal-technically coupled to the control device for detecting the closed eyelid, wherein the control unit is designed to activate the irradiation device only when the eyelid sensor transmits a signal indicating a closed eyelid. For this purpose, the sensor may use various physical properties and include electro-chemical, mechanical or optical elements to measure, for example, skin resistance/conductivity, contact pressure, or reflectivity differences. According to this embodiment, the device comprises an eyelid sensor adapted to detect whether the patient's eyelid is closed or open. Such an eyelid sensor can be designed in different ways, for example, a heat transfer can be measured starting from the concave contact surface and, based on this heat transfer, it can be determined whether the planar contact to the closed eyelid is present or not. The sensor can also measure the conductivity of the tissue surface on which the lens element rests and conclude from this whether this tissue surface is the surface of the closed eyelid. The eyelid sensor may also be designed as an image capture unit with image evaluation to detect a closed or opened eyelid. The eyelid sensor can also be designed as a light barrier, for example, which can differentiate the reflection of the eyelid or the surface of the eye from each other and in this way can detect the closed eyelid. The eyelid sensor is signal-technically coupled with the control device. This ensures that the control device can control the exposure to irradiation depending on the signal from the eyelid sensor. In this way, for example, it can be avoided that too much irradiation acts on the eye if the eyelid is not closed, in order to avoid an overdose of therapeutic irradiation.

Alternatively, or in addition to such an eyelid sensor, the lens element may also be configured to provide a mechanical blockage of the closed eyelid, thereby preventing the eyelid from opening when the device is in use. This can be achieved, for example, by the concave contact surface being designed for positive contact on the eyelid and then blocking the eyelid opening movement in such positive contact.

The device may be further embodied by an input user interface, a graphical output user interface for outputting graphical information, wherein the control unit is programmed to display in an information step to the patient information on a subsequent therapeutic treatment step via the graphical user interface, and to perform the treatment step after the information step, wherein the irradiation duration and irradiation intensity correspond to a treatment mode entered via the input user interface and/or a treatment mode output via the graphical output user interface. According to this embodiment, the device according to the invention also comprises an input and output user interface, which may be, for example, a touch screen, a keyboard and a screen, a voice recognition and voice output or the like, and combinations thereof. Via these interfaces, the user can, on the one hand, enter data concerning the therapy and, on the other hand, information concerning the therapy can be output to the user. The further development is particularly suitable for making the device suitable for home use by the user and for making it possible to make safety-related inputs and outputs via the interfaces in order to enable the device to be used even without medically trained personnel.

It is particularly preferred if the control unit is programmed to request user input via the input user interface in an input step between the information step and the treatment step and to start the treatment step after receiving a predetermined user input. Such input by the user after the information step and before the treatment step can provide a safety prompt and confirmation to the user and thus prevent a mishandling or a faulty start in the treatment.

It is even more preferred that the control unit is programmed to receive user-related personal information via the input user interface, to identify a user identity from a user identity data memory for the control unit based on this user-related personal information, and to perform a predetermined treatment step stored for this user identity in a treatment programme data memory in dependence on this user identity. According to this embodiment, a user identity is determined before the treatment starts. In particular, this ensures that the treatment is only performed on the intended patient and no other person. The user identity can be determined, for example, by means of a password, a fingerprint, iris recognition, or the like. In particular, it is preferred if an eyelid sensor, which is designed to detect an open or closed eyelid, is also used at the same time to determine a user identity on the basis of an iris recognition.

In this context, it is particularly preferred if the input user interface comprises a digital image capture unit and the control unit is programmed to receive image data from the digital image capture unit and to determine the user identity on the basis of the image data, in particular, to determine the user identity on the basis of image data describing an iris geometry of the user on the basis of an iris recognition.

Still further, it is preferred to continue the device according to the invention by arranging the control unit and the graphical output user interface in the irradiation base device, and the irradiation base device further comprises a irradiation directing device for directing the irradiation emitted by the irradiation source to the optical lens element held on the holding and positioning device in front of the patient's eye. According to this embodiment, the control unit and graphical output user interface are arranged in the irradiation base device and are thus integrally designed in one of possibly only two structural units of the device according to the invention. This achieves on the one hand a compact construction and on the other hand a device that is easy to handle.

Furthermore, it is preferred if the control device is formed in a smart tablet, laptop, or smartphone. According to this embodiment, the control unit can be realised by software that is executed, for example, as an application on such a smartphone, smart tablet or laptop.

It is even more preferred if the holding and carrying device has a total weight and dimensions that allow the device to be held on the patient's head by the carrying device alone. According to this embodiment, comfortable wearing of the device according to the invention on the patient's head is possible and the patient can also move with the device in place, change his position, and thereby establish a comfortable treatment situation.

It is further preferred if the irradiation device comprises two beam exit directions spaced apart from each other for simultaneous irradiation of both eyes of the patient. This makes it possible to treat both eyes at the same time, thus shortening the treatment time. It can be provided that the therapeutic irradiation for both eyes is derived from a single irradiation source and distributed to both eyes by appropriate beam splitting. Alternatively, two different irradiation sources can be used, whereby each irradiation source is intended for one eye.

The irradiation can be designed by a beam splitter or at least 2 independent beam sources so that one eye or both eyes can be treated simultaneously. For this purpose, the eyes are separated from each other by an optical separation. The irradiation parameters and the irradiation frequency of both eyes can be designed differently. This allows the patient to maintain a consistent routine over a treatment period of several days, performing a treatment procedure every morning. The irradiation can then be carried out by the control unit in such a way that the eyes are irradiated in the same way or differently. For example, one eye can be irradiated only every two days with the corresponding effective therapy wavelength (or other parameters), while the other eye is also irradiated, but not with a therapeutically effective irradiation dose, for example, a therapeutically ineffective wavelength within the visible spectrum or with a therapeutically ineffective irradiation intensity. In this way, a kind of placebo effect can also be used in a targeted manner.

Still further, it is preferred that the irradiation device comprises a first optical system comprising at least a first optical lens, a first optical collimator and/or a first optical filter unit, and a second optical system spaced from the first optical system along an irradiation path axis and comprising at least a second optical lens, a second optical collimator, and/or a second optical filter unit. According to this embodiment, the irradiation device is designed to irradiate the two eyes of the patient simultaneously and, for this purpose, corresponding optical devices are provided for each of the two eyes. In particular, this also makes it possible to treat the two eyes with different therapeutic irradiation, for example, in terms of frequency, irradiation intensity, or other irradiation parameters.

It is even more preferred if the control unit is programmed to store and control a treatment plan comprising at least a first and a second treatment procedure for a user, and to control a first treatment procedure for the user, to store the completion time of the first treatment procedure, to start the second treatment procedure under the condition that a minimum period of time has elapsed since the completion time of the first treatment procedure. According to this form of further development, the control unit is programmed to carry out an intelligent and at the same time error-proof treatment procedure over a longer period of time and also to safely carry out several individual treatment steps in a temporally spaced manner. In this case, appropriate programming ensures that two treatment steps or treatment measures are not carried out at such a short time interval from each other that overdosed irradiation could damage the eye or at least jeopardise the success of the therapy. This is achieved by programming the system for a predetermined minimum period of time and not allowing subsequent irradiation if this minimum period of time is not reached.

Still further, it is preferred to advance the apparatus by programming the control unit to receive, via the input user interface, diagnostic data characterising a body condition, to generate, after a treatment procedure, therapy data characterising the treatment procedure performed, to transmit, via a data transmission unit, a data packet, comprising the diagnostic data and the therapy data to a receiving device of an expert computer via a data transmission unit, to receive instruction data from a transmitting device of the expert computer via the data transmission unit, and to control the irradiation apparatus for carrying out a treatment procedure characterised by the instruction data. According to this embodiment, the device is capable of receiving diagnostic data on the one hand, generating therapy data on the other hand, and transmitting these data via a data transmission unit. It is thus made possible to exchange relevant data directly related to a treatment carried out or to be carried out by the device with an expert computer, which is monitored and operated by a medically trained person, for example, and in this way to enable monitoring of the treatment progress, the treatment success. This advanced training is particularly advantageous for the particular suitability of the device according to the invention for home use by the patient. The further development also makes it possible to control treatment procedures on the device from the expert computer, thus enabling a treating physician to remotely control such treatment procedures.

Still further, it is preferred that the control unit is programmed to receive, via the input user interface, diagnostic data characterising a body condition, compare the diagnostic data with predetermined body condition data stored in an electronic data memory of the control unit, select a subsequent treatment procedure from a plurality of treatment procedures stored in the electronic data memory of the control unit based on a match with one of the stored body condition data, the exceeding or falling below of a body condition defined by the body condition data, to select a subsequent treatment procedure from a plurality of treatment procedures stored in the electronic data memory of the control unit, and to control the irradiation device to perform the selected treatment procedure. According to this embodiment, the control device is programmed to detect, on the basis of diagnostic data received, for example by being manually entered or by being analysed from an image capture device by image evaluation, a change in the patient's characteristics describing the state of health and, in dependence thereon, to select the further treatment steps. In this way, the further treatment can be designed depending on the progress of the treatment in the sense of partial successes achieved, and in this case, for example, the appropriate treatment step for the further treatment can be selected from several different treatment steps available for selection and then carried out. It is also possible to select the treatment step that has achieved the best result based on a comparison of therapy successes achieved with different treatment steps and then continue the treatment with this treatment step.

It is further preferred if the input user interface comprises a digital image capture device and the diagnostic data includes image capture data describing the image of a treated tissue. According to such an embodiment, a digital image capture device performs an image capture comprising the image of the retinal tissue and thereby enables a direct analysis of the treatment result by corresponding image evaluation. Hereby, the success of certain treatment steps can be directly evaluated and, on the basis of such an evaluation, the planning of further treatment steps, such as the selection of a certain, successful treatment step, can also be carried out.

### BRIEF DESCRIPTION OF THE DRAWINGS

Preferred embodiments of the invention are explained with reference to the accompanying figures. They show:

FIG. 1 is a schematic top view of a first embodiment of the invention;

FIG. 2 is a schematic top view of a second embodiment of the invention; and

FIG. 3 is a schematic side view of a third embodiment of the invention.

### DETAILED DESCRIPTION OF THE EMBODIMENTS

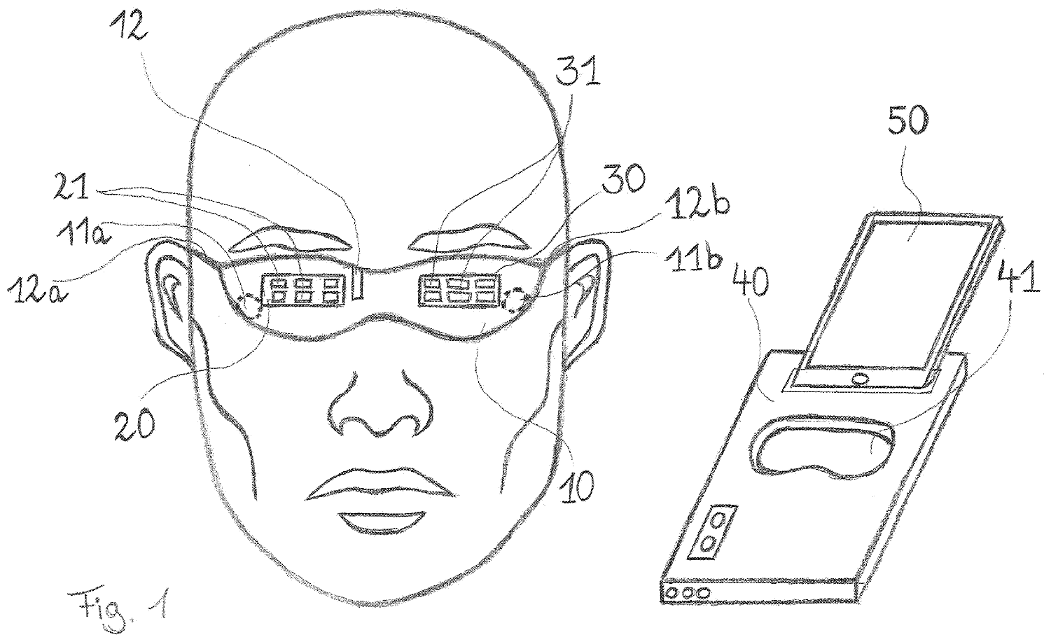

Referring first to FIG. 1, a patient wears the device according to the invention as a face mask 10, with eyes closed and held in position in front of the eyes by means of spectacle-like holding brackets 12a, 12b as a holding device. The face mask 10 comprises, in the axial direction of the eyes with the face mask 10 in place, one right and one left lens element 20, 30 for each of the right and left eyes. Six right and six left irradiation sources 21, 31 are integrated into each of these lens elements 20, 30. Each of these irradiation sources is integral with a sensor which detects the reflection of the irradiated irradiation and compares it with reference values which a control unit 12 integrated in the mask 10 can use to determine whether the lid of the respective eye is closed or open.

The device according to this embodiment further comprises a base station 40 into which the face mask 10 can be inserted in an adapter 41 in order to charge rechargeable batteries 11a, 11b arranged in the face mask 10 by means of a corresponding contact. The base station 40 or the face mask 10 is further coupled to a smartphone 50. The smartphone 50 is thus in signal connection with a receiver integrated in the control unit 12 in the face mask 10 by means of a Bluetooth® connection. A corresponding control software on the smartphone 50 can be operated via the user interface of the smartphone 50 and controls the irradiation process through the irradiation sources in the lens elements 20, 30.

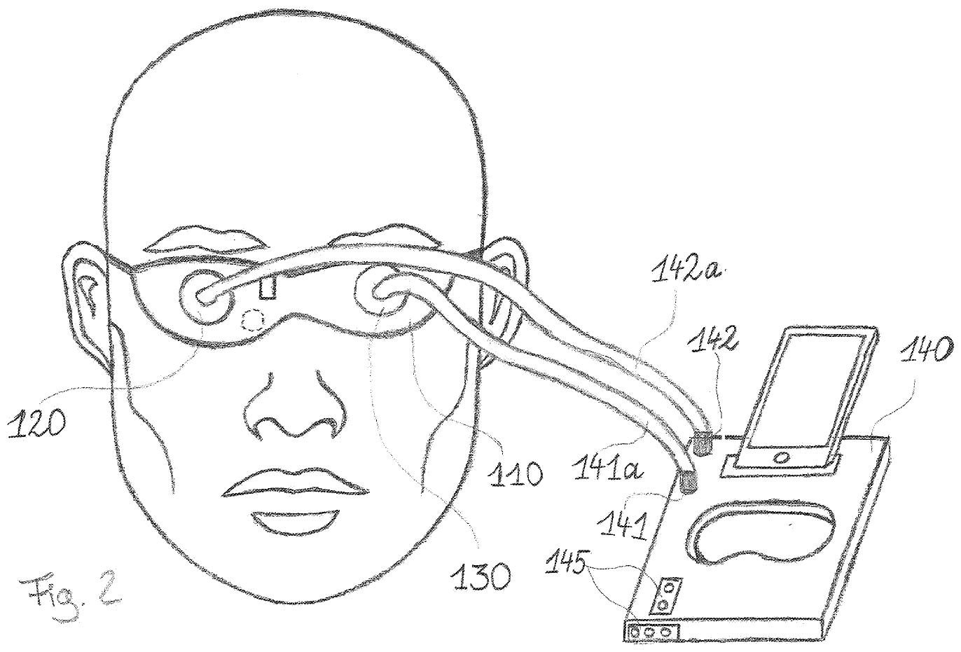

FIG. 2 shows a second embodiment of the invention. In this embodiment, a face mask 110 is also placed on the eye and comprises corresponding lens elements 120, 130 in the axial direction of the right eye and the left eye. However, in this case, the lens elements 120, 130 do not comprise irradiation sources on the face mask 110 itself. Instead, one irradiation source 141, 142 each is provided in a base station 140, which is connected to the face mask 110 via a light guide 141a, 142a. The irradiation sources 141, 142 in the base station 140 emit irradiation which is guided via the light guides 141a, 142a to the face mask 110, where it is guided to the two lens elements 120, 130 and coupled into them, thereby effecting therapeutic irradiation of the retina.

The base station 140 of the second embodiment further comprises its own user interface 145, via which the patient can operate the device, in particular set irradiation parameters such as irradiation intensity and irradiation duration.

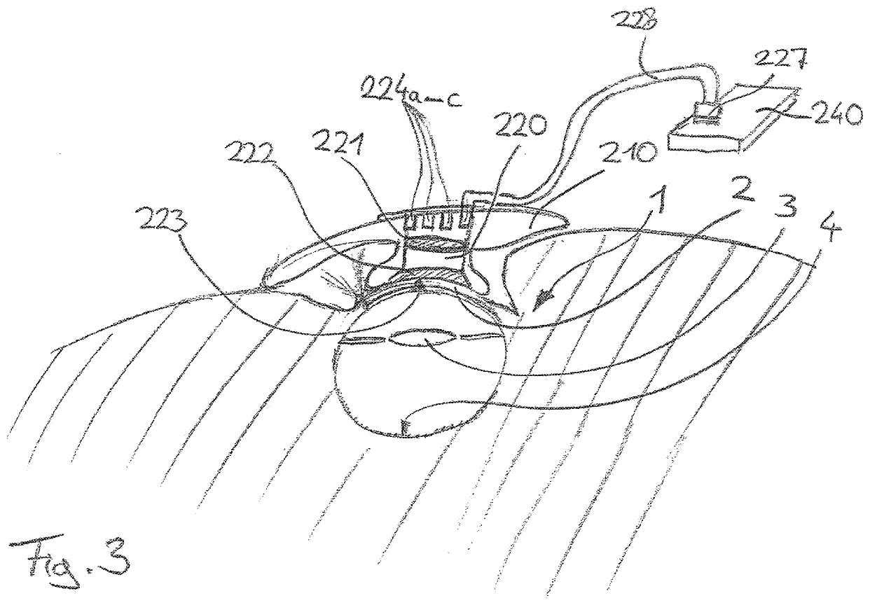

FIG. 3 shows a partially cut side view of a third embodiment of the invention. In this third embodiment, a face mask 210 is also applied to the face. The face mask 210 comprises a lens element 220 which comprises an imaging lens 221 and a diffuser 222 which has a concave bearing surface 223 directed towards the closed eyelid 2. This concave bearing surface 223 rests directly on this closed eyelid and couples a irradiation into the eyelid. This irradiation penetrates the eyelid and reaches the retina 4 of the eye 1 through the eye lens 3 to achieve a therapeutic effect there.

In the face mask 210, irradiation sources 224a-d are arranged immediately above the diffuser 222, which emit irradiation into the diffuser 222, which is irradiated into the eyelid via the concave surface 223 and from there reaches the retina.

Furthermore, in this embodiment, an external irradiation source 227 is present, which is connected to the face mask 210 via a light guide 228. The irradiation emitted by the external irradiation source 227 is coupled into the lens element 220 via this light guide 228 and also enters the diffuser 222 to reach the retina via the concave surface 223.

The external irradiation source 227 is arranged in a base station 240 which, as in the first embodiment, comprises a receptacle with an electrical contact element for charging an energy storage device in the face mask 210.

1.-26. (canceled)

27. A device for the therapeutic treatment of the retina of an eye of a patient, comprising:
at least one irradiation source which emits a therapeutically effective irradiation;
a control unit for driving the irradiation source, the control unit being programmed to deliver the therapeutically effective irradiation with a predetermined irradiation duration and irradiation intensity from the irradiation source to the retina of the eye of the patient; and
a holding and positioning device for holding an optical lens element in a defined position and orientation in front of the eye of the patient, the optical lens element having a irradiation-transmitting concave surface for resting on a closed eyelid of the patient, and the irradiation source cooperating with the optical lens element to deliver the therapeutically effective irradiation to the retina via the optical lens element.

28. The device according to claim 27, wherein the optical lens element comprises a diffuser adapted to distribute the therapeutically effective irradiation from the irradiation source onto the irradiation-transmitting concave surface.

29. The device according to claim 28, wherein the diffuser is designed as an integrating sphere or as a layer of a semitransparent material.

30. The device according to claim 27, wherein the optical lens element comprises a thermal heating or cooling device.

31. The device according to claim 27, further comprising a set of contact surface elements, one of which is attachable to the concave surface of the optical lens element, the set comprising:
a first contact surface element having a first refractive index; and
a second contact surface element having a second refractive index different from the first refractive index.

32. The device according to claim 27, wherein the holding and positioning device comprises a partial face mask which at least partially covers an eye area of a face of the patient.

33. The device according to claim 32, wherein the partial face mask is held in a predetermined position on the patient's face by:
a band passed around the head;
at least one retaining element comprising a strap or spectacle temples passed around the ears; or
suction cups is held in a predetermined position on the patient's face.

34. The device according to claim 28, wherein the irradiation source is fixed to the holding and positioning device and lies in a treatment position defining an irradiation axis aligned with a pupil of the patient and the diffuser.

35. The device according to claim 34, further comprising a second irradiation source arranged at a distance from the holding and positioning device on an irradiation base device and wherein the therapeutically effective irradiation is guided from the irradiation base device to the holding and positioning device by a flexible irradiation guide.

36. The device according to claim 27, wherein the irradiation source is arranged at a distance from the holding and positioning device on an irradiation base device and the therapeutically effective irradiation is guided from the irradiation base device to the holding and positioning device by means of a flexible irradiation guide.

37. The device according to claim 27, wherein the concave surface is designed for superficial contact with the eye of the patient or the closed eyelid of the patient over a contact area of at least 1 cm².

38. The device according to claim 27, wherein the lens element has an eyelid sensor in signal communication with the control unit for detecting the closed eyelid of the patient, and the control unit is adapted to activate the irradiation device only when the eyelid sensor transmits a signal signalling the closed eyelid of the patient.

39. The device according to claim 27, further comprising a base station comprising a receiving device for the holding and positioning device, the receiving device further comprising a heating or cooling device for heating or cooling the optical lens element.

40. The device according to claim 27, further comprising:
an input user interface; and
a graphical output user interface for outputting graphical information;
wherein the control unit is programmed to, in an information step, display to the patient information on a subsequent therapeutic treatment step via the graphical output user interface, and performing the subsequent therapeutic treatment step after the information step, wherein the irradiation duration and irradiation intensity correspond to a treatment mode input via the input user interface and/or an output via the graphical output user interface.

41. The device according to claim 40, wherein the control unit is programmed to, between the information step and the subsequent therapeutic treatment step, in an input step request a user input via the input user interface, and start the subsequent therapeutic treatment step after receiving a predetermined user input.

42. The device according to claim 40, wherein the control unit is programmed to:
receive user-related personal information via the input user interface;
identify a user identity from a user identity data stored by the control unit on the basis of this user-related personal information; and
perform a predetermined treatment step stored for that user identity in a treatment programme data memory as a function of the user identity.

43. The device according to claim 42, wherein:
the input user interface comprises a digital image capture unit, and
the control unit is programmed to receive image data from the digital image capture unit and to determine the user identity on the basis of image data describing an iris geometry of the patient on the basis of an iris recognition.

44. The device according to claim 40, wherein the control unit and the graphical output user interface are arranged in the irradiation base device, and the irradiation base device further comprises a irradiation guide device for directing the therapeutically effective irradiation emitted from the irradiation source to the optical lens element held on the holding and positioning device in front of the patient's eye.

45. The device according to claim 27, wherein the control unit is provided in a smart tablet, laptop, or smartphone.

46. The device according to claim 27, wherein the holding and positioning device has a total weight and dimensions allowing the device to be held to the patient's head by the holding and positioning device alone.

47. The device according to claim 27, wherein the irradiation device comprises two beam exit directions spaced apart from each other for simultaneous irradiation of both eyes of the patient.

48. The device according to claim 27, wherein the irradiation device further comprises:
a first optical system comprising:
at least a first optical lens;
a first optical collimator; or
a first optical filter unit; and
a second optical system spaced from the first optical system along a irradiation trajectory axis comprising:
at least a second optical lens;
a second optical collimator; or
a second optical filter unit.

49. The device according to claim 27, wherein the control unit is programmed to:
store and control a treatment plan comprising at least a first and a second treatment operation for a user, and to:
control a first treatment procedure for the patient;
save the completion time of the first treatment procedure; and
start a second treatment procedure on the condition that a minimum period of time has elapsed since the completion time of the first treatment procedure.

50. The device according to claim 40, wherein the control unit is programmed to:
receive a diagnostic data characterising a body condition of the patient via the input user interface;
generate a therapy data after a treatment procedure which characterises the treatment procedure carried out;
send a data packet comprising the diagnostic data and the therapy data to a receiving device of an expert computer via a data transmission unit;
receive an instruction data from a transmitting device of the expert computer via the data transmission unit; and
control the irradiation device to perform a treatment procedure characterised by the instruction data.

51. The device according to claim 50, wherein the control unit is programmed to:
receive the diagnostic data characterising the body condition via the input user interface;
compare the diagnostic data with a predetermined body condition data stored in an electronic data memory of the control unit;
select a subsequent treatment procedure from a plurality of treatment procedures stored in the electronic data memory of the control unit on the basis of the correspondence with one of the stored body condition data exceeding or the falling below of a body condition defined by the body condition data; and
control the irradiation device to perform the selected treatment procedure.

52. The device according to claim 50, wherein the input user interface comprises a digital image capture device and the diagnostic data includes image capture data describing the image of a treated tissue.
